(19) United States
(12) Patent Application Publication
(10) Pub. No.: US 2024/0009150 A1
(43) Pub. Date: Jan. 11, 2024

(54) CAPSAICYNS IN THE TREATMENT OF LEAKY GUT

(71) Applicant: AXICHEM AB [SE]/[SE], Oslo (NO)

(72) Inventors: Erik LAGER, Lund (SE); Lucas ALTEPOST, Nesøya (NO); Torsten HELSING, Kleppestø (NO); Bomi FRAMROZE, Menlo Park, CA (US)

(21) Appl. No.: 18/252,717

(22) PCT Filed: Nov. 16, 2021
(86) PCT No.: PCT/EP2021/081795

§ 371 Date: May 11, 2023

(30) Foreign Application Priority Data
Nov. 17, 2020 (NO) 20201253

(51) Int. Cl. A61K 31/165; A61P 31/04; A61P 1/00

(52) U.S. Cl. CPC A61K 31/165; A61P 31/04; A61P 1/00

(57) ABSTRACT

The present invention relates to the treatment of leaky gut. More particularly, the invention provides compounds, and compositions thereof, for use in methods for the treatment of leaky gut and in methods for the treatment of a subclinical increase in gut permeability. The invention further provides compounds, and compositions thereof, for use in methods for the treatment of leaky gut syndrome. The invention further provides compounds, and compositions thereof, for use in methods for the improvement of at least one symptom associated with leaky gut syndrome. The present invention also relates to a method for treatment of leaky gut, a method for treatment of leaky gut syndrome, and a method for improving at least one symptom associated with leaky gut syndrome.

### FIELD OF THE INVENTION

The present invention relates to the treatment of leaky gut. More particularly, the invention provides compounds, and compositions thereof, for use in methods for the treatment of leaky gut and in methods for the treatment of a subclinical increase in gut permeability. The invention further provides compounds, and compositions thereof, for use in methods for the treatment of leaky gut syndrome. The invention further provides compounds, and compositions thereof, for use in methods for the improvement of at least one symptom associated with leaky gut syndrome. The present invention also relates to a method for treatment of leaky gut, a method for treatment of leaky gut syndrome, and a method for improving at least one symptom associated with leaky gut syndrome.

### BACKGROUND OF THE INVENTION

The gastrointestinal tract, i.e. the tract from the mouth to the anus present in all vertebrates and most invertebrates, includes all the organs of the digestive system. It is the most extended barrier that separates the body's internal and external environment; the lumen of the gastrointestinal tract tube is outside of the body of the animal, with the cells lining the tract creating a barrier between the body and the external world. Thus, while the primary role of the gastrointestinal tract is extraction of nutrients, it also forms an important part of the immune system, preventing pathogens such as viruses, molds, and bacteria from entering the blood and lymph circulatory systems.

It is well known that for critically ill patients, loss of gut integrity can result in the movement of bacteria into the circulation, which can ultimately lead to multiple organ failure and death. Lately, there has been an increasing focus on the importance of gut barrier integrity in subjects that are not critically ill. There are indications that toxins may cross the epithelial barrier from the gut lumen into the host, resulting in local and systemic immune responses. Different pathways, including increased intestinal mucosal paracellular permeability, have been discussed. Such subclinical increases in gut permeability are often referred to as leaky gut, and the various health problems thought to be caused by this phenomenon are collectively termed leaky gut syndrome.

The intestines are home to the wide range of bacteria called gut microbiota, comprising some 4,000 different strains of bacteria having diverse roles in maintenance of immune health and metabolism. It is likely that leaky gut syndrome involves imbalances in gut microbiota, one theory suggesting that the imbalances in the microbiota triggers the body's immune response, which again results in gut inflammation and increased intestinal permeability. The frequently suggested risk factors for leaky gut syndrome, including physiologic stressors such as anxiety, the use of non-steroidal anti-inflammatory drugs (NSAIDs), alcohol consumption, and dietary components including emulsifiers and other food additives, lend credence to the theory of involvement of the microbiota in leaky gut, as these factors also are known to affect the balance of the gut flora.

It is believed that a leaky gut leads to enhanced entry of pathogenic bacteria and bacterial toxins into the systemic circulation, with repeated occurrences provoking systemic inflammation and triggering numerous diseases. Even certain bacteria normally present in a healthy gastrointestinal tract are believed to become pathogenic during leaky gut. There are indications that quorum sensing between bacteria in the gut may be a major factor in leaky gut, affecting its onset and development.

It is widely accepted that increased intestinal permeability is associated with certain gastrointestinal conditions such as celiac disease, Crohn's disease, and irritable bowel syndrome (IBS), and inflammatory bowel disease (IBD). It is not, however, clear whether leaky gut is a cause or a symptom of any of these conditions, or if the observed link can be explained by leaky gut syndrome frequently occurring simultaneously with said conditions or diseases. Further, it has been suggested that leaky gut or leaky gut syndrome also may be linked to a wide range of other illnesses, including autoimmune diseases (lupus, type 1 diabetes, multiple sclerosis, autoimmune hepatitis), chronic fatigue syndrome, fibromyalgia, arthritis, allergies, asthma, polycystic ovary syndrome, obesity, and even autism and mental illness. Such causes and effects have yet to be confirmed in clinical studies in humans.

In addition to the possible contributions to other illnesses, leaky gut syndrome is thought to cause symptoms including chronic diarrhoea, constipation, bloating, gas and stomach cramps; skin problems such as acne, rashes, and eczema; food sensitivities and nutritional deficiencies; aches and pains including joint pain; headaches, confusion, concentration difficulties, and fatigue. The symptoms may be contributed to chronic inflammation throughout the body.

While treatments such as various restrictive diets, dietary supplements, and probiotics have been suggested, there is little evidence that the treatments offered against leaky gut are of benefit. Antibiotics have not been found useful; in fact, it appears that a leaky gut can be made worse by antibiotics. None of the suggested treatments have been adequately tested to determine if they are safe and effective for this purpose, and there is a lack of official recommendations regarding regimes for treating or managing the symptoms of leaky gut syndrome. A need therefore exists for new compounds and methods for the treatment of leaky gut.

### BRIEF SUMMARY OF THE INVENTION

The inventors have discovered that compounds of formula I can positively impact leaky gut, and thus that these compounds are useful in methods for the treatment of leaky gut and/or leaky gut syndrome, and in methods for the improvement of at least one symptom associated with leaky gut syndrome. They have found that these compounds inhibit quorum sensing, which is believed to be a major factor in leaky gut—there are strong indications that inhibiting bacterial quorum sensing may improve intestinal barrier function, by preventing the signalling that promotes individual bacteria to become pathogenic by, for example, forming tertiary structures such as biofilms, thus positively affecting leaky gut. The compounds of formula I represent promising candidates for treatment of leaky gut, leaky gut syndrome, and symptoms associated with leaky gut syndrome.

In one aspect, the present invention provides a compound of formula I, or a pharmaceutically acceptable salt or solvate thereof,

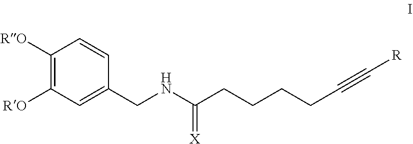

wherein
   X is selected from oxygen and sulphur,
   R is selected from the group comprising cyclohexyl, phenyl and substituted phenyl;
   wherein said substituted phenyl is substituted in any one or more positions with 1-5 identical or different substituents selected from the group comprising fluoro; chloro; bromo; iodo, cyano, nitro, trifluoromethyl, C₁-C₆ straight chain and branched alkoxy, C₁-C₆ sulfoxy, —S—C₁-C₆ alkyl, C₁-C₆ straight chain and branched alkyl, alkenyl, and alkynyl C₂-C₆ straight chain and branched alkenyl, C₂-C₆ straight chain and branched alkynyl, C₁-C₆ fluoroalkyl, chloroalkyl, bromoalkyl, and iodoalkyl, COO—C₁-C₆ alkyl, O(CO)—C₁-C₆ alkyl, NH—C₁-C₆ alkyl, N(C₁-C₆ alkyl)₂, CON(C₁-C₆ alkyl)₂, and NH(CO)—C₁-C₆ alkyl;
   R′ is selected from the group comprising hydrogen, C₁-C₆ straight chain and branched alkyl, and C₃-C₆ cycloalkyl, and
R″ is selected from the group comprising hydrogen, benzyl, and acetyl, for use in a method for the treatment of leaky gut.

In another aspect, the invention provides a compound of formula I, for use in a method for the treatment of a subclinical increase in gut permeability in a subject who experiences one or more symptoms associated with leaky gut syndrome and who displays a significant deviation in the level of at least one biomarker associated with leaky gut, leaky gut syndrome and/or intestinal hyperpermeability compared to a normal level of the at least one biomarker.

In another aspect, the invention provides a composition comprising a compound of formula I, or a pharmaceutically acceptable salt or solvate thereof, for use in a method for the treatment of leaky gut.

In another aspect, the invention provides a method for the treatment of leaky gut, a method for the treatment of leaky gut syndrome, or a method for the improvement of at least one symptom associated with leaky gut syndrome, the method comprising the step of administering an effective amount of a compound of formula I or a pharmaceutically acceptable salt or solvate thereof, or a composition comprising an effective amount of a compound of formula I or a pharmaceutically acceptable salt or solvate thereof, to a subject.

In another aspect, the invention provides the use of a compound of formula I or a pharmaceutically acceptable salt or solvate thereof, or a composition comprising an effective amount of a compound of formula I or a pharmaceutically acceptable salt or solvate thereof, to inhibit quorum sensing, to inhibit quorum sensing in the gut, to treat leaky gut, to treat leaky gut syndrome, or to improve at least one symptom associated with leaky gut syndrome.

In another aspect, the invention provides a method for the treatment of leaky gut, leaky gut syndrome, or increased intestinal permeability, the method comprising the step of administering to a subject an effective amount of a compound of formula I, or a pharmaceutically acceptable salt or solvate thereof.

In another aspect, the invention provides the use of a compound of formula I, or a pharmaceutically acceptable salt or solvate thereof, for inhibition of quorum sensing.

### BRIEF DESCRIPTION OF THE DRAWINGS

FIG. 1 shows time-kill curves of different concentrations of phenylcapsaicin against Salmonella ATCC14028.

FIG. 2 shows time-kill curves of different concentrations of phenylcapsaicin against Listeria monocytogenes ATCC11915.

FIG. 3 shows time-kill curves of different concentrations of phenylcapsaicin against Campylobacter jejuni ATCC11168.

### DETAILED DESCRIPTION OF THE INVENTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

The term “capsaicyn” as used herein refers to hept-6-yn derivatives of capsaicin ((E)-N-(4-hydroxy-3-methoxybenzyl)-8-methylnon-6-enamide), of general formula a, wherein Y represents any substituent and Z represents any one or more substituents in any one or more positions on the phenyl ring.

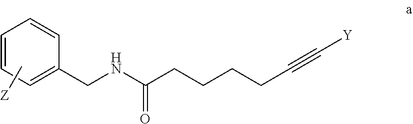

The reader should note that the nomenclature used for capsaicin derivatives in the art is not consistent, and that while the term capsaicyns is used, for ease of reading, when reference is made to the general group of compounds, the well-established common name “phenylcapsaicin” will be used when referring to N-[(4-hydroxy-3-methoxyphenyl)methyl]-7-phenylhept-6-ynamide (structure V), and “phenylcapsaicins” will be used to refer to derivatives thereof.

The term “thioamide capsaicyn” as used herein refers to hept-6-yn derivatives of capsaicin comprising a thioamide group rather than the amide group of capsaicin, of general formula b, wherein Y represents any substituent and Z represents any one or more substituents in any one or more positions on the phenyl ring.

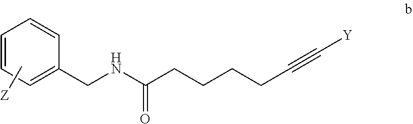

As will be understood, any of the compounds herein described may be provided in the form of a pharmaceutically acceptable salt or solvate thereof. Procedures for salt formation and solvate formation are conventional in the art.

As used herein, the terms straight chain and branched alkyl, alkenyl, alkynyl, alkoxy comprise all such substituents of a given chain length, including substituents that are cyclic or comprise a cycle.

The term “derivative” as used herein refers to a molecule that differs in chemical structure from a parent compound. Examples of derivatives include, without limitation: homologues, which differ incrementally from the chemical structure of the parent, such as a difference in the length of an aliphatic chain; molecular fragments; structures that differ by one or more functional groups from the parent compound, such as can be made by transforming one or more functional groups of a parent; a change in ionization state of a parent, such as ionising an acid to its conjugate base; isomers, including positional, geometric and stereoisomers; and combinations thereof.

The terms “treating” and “treatment” and “therapy” (and grammatical variations thereof) are used herein interchangeably, and refer to 1) inhibiting the disease; for example, inhibiting a disease, condition or disorder in a subject who is experiencing or displaying the pathology or symptomatology of the disease, condition or disorder, including prevention of disease (i.e. prophylactic treatment, arresting further development of the pathology and/or symptomatology), or 2) alleviating the symptoms of the disease, or 3) ameliorating the disease; for example, ameliorating a disease, condition or disorder in an subject who is experiencing or displaying the pathology or symptomatology of the disease, condition or disorder (i.e., reversing the pathology and/or symptomatology). The terms may relate to the use and/or administration of medicaments, active pharmaceutical ingredients (API), food additives, food supplements, dietary supplements, nutritional supplements, over-the-counter (OTC) supplements, medical foods, and/or a pharmaceutical grade supplements.

As used herein, the term “composition” refers to a mixture, in any formulation, of one or more compounds according to the invention with one or more additional chemical component.

As used herein, the terms “administer”, “administration”, and “administering” refer to (1) providing, giving, dosing and/or prescribing by either a health practitioner or their authorised agent or under their direction, or by self-administration, a formulation, preparation or composition according to the present disclosure, and (2) putting into, taking or consuming by the subject themselves, a formulation, preparation or composition according to the present disclosure.

As used herein, “subject” means any human or non-human animal selected for treatment or therapy, and encompasses, and may be limited to, “patient”. None of the terms should be construed as requiring the supervision (constant or otherwise) of a medical professional (e.g., physician, nurse, nurse practitioner, physician's assistant, orderly, clinical research associate, etc.) or a scientific researcher.

The term “therapeutically effective dose” as used herein means the amount of compound according to the invention which is effective for producing the desired therapeutic effect in a subject at a reasonable benefit/risk ratio applicable to any treatment. The therapeutically effective dosage amount may vary depending upon the route of administration and dosage form.

As used herein, the term “pharmaceutically acceptable” means that compound must be physiologically acceptable to the recipient as well as, if part of a composition, compatible with other ingredients of the composition.

As used herein, the term “prodrug” means any compound which under physiological conditions is converted into any of the compounds according to the invention.

The term “symptom”, as used herein, refers to any subjective or objective evidence of disease, any physical disturbance, or any morbid phenomenon or departure from the normal in structure, function, or sensation observed in or by a subject. As used herein, the term “improvement of a symptom”, or any variations thereof, refers to an alleviation or amelioration of said symptom.

As used herein, the term “leaky gut” refers to subclinical increases in gut permeability. The symptoms caused by leaky gut is referred to as “leaky gut syndrome”.

Bacteria become pathogenic and significantly increase in numbers in a host by quorum sensing (QS). Quorum sensing is a cell-to-cell communication mechanism leading to differential gene expression in response to high population density. Via this process a particular species of bacteria forms biofilms and the individual cells begin to act as one larger tertiary organism. This process is mediated via a cell density dependent expression of hormone-like compounds called autoinducers. Several processes responsible for successful establishment of bacterial infection are mediated by quorum sensing and the expression of virulence genes. Inhibition of quorum sensing is therefore considered a suitable target for reducing the population of pathogenic bacteria within a host without any anti-microbial action.

The link between quorum sensing and biofilm formation is well-established, see e.g. the review article “Sociomicrobiology: the connections between quorum sensing and biofilms” from 2005 (Parsek and Greenberg, Trends Microbiol. 2005, 13(1), 27).

Based on the premise that leaky gut syndrome involves imbalances in gut microbiota, the theory that leaky gut may be treated by limiting quorum sensing has lately gained increasing support. Targeting bacterial quorum sensing has shown promise in improving intestinal barrier function, lending credence to the theory. For example, in (Mol. Med. Rep. 2019, 19, 4057), Adiliaghdam et al., titled demonstrate a robust relation between the inhibition of a crucial P. aeruginosa quorum sensing system and the regulation of the intestinal integrity following thermal injury and burn wound infection. The results disclosed therein provide evidence that silencing of the P. aeruginosa quorum sensing transcription factor MvfR leads to a less prominent induction of gut barrier damage, and the results presented in the article indicate that the inhibition of this quorum sensing system mitigates gut hyperpermeability.

Hept-6-yne derivatives of capsaicin, herein referred to as capsaicyns, are valuable compounds with various potential uses. Set apart from the natural compound capsaicin by their alkyne moiety replacing the alkene moiety of capsaicin, these synthetic capsaicin derivatives have found their use in various areas, including food industries, agriculture, pharmacology, and marine antifouling paint. The perhaps most widely used derivative, phenylcapsaicin (V), has been shown to have low systemic toxicity and to be safe with regards to gene mutations and chromosomal damage (Rage Paulsen et al., Toxicology Research and Application 2018, 2, 1), and has been examined by the European Food Safety Authority and regarded as safe (EFSA NDA Panel et al., EFSA Journal 2019, 17(6), e05718).

Natural capsaicin is widely suspected of causing or contributing to leaky gut and leaky gut syndrome, and sufferers are frequently advised to avoid foods that contain capsaicin.

Surprisingly, the inventors have found that compounds of structure I are useful in a method for the treatment of leaky gut, a method for the treatment of leaky gut syndrome, or a method for the improvement of at least one symptom associated with leaky gut syndrome.

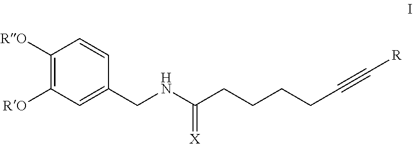

The inventors' experiments have indicated that said compounds are quorum sensing inhibitors in a dose dependent manner at doses lower than the sub-lethal antibacterial doses. Specifically, phenylcapsaicin (V) at doses of 61.5, 31.25, 15.625, and 7.81 μg/ml showed strong to weak quorum sensing inhibiting activity in a dose responsive manner, while showing antibacterial activity at higher doses. (500/250/125 μg/ml).

The natural compound capsaicin did not exhibit quorum sensing inhibiting activity at non antibacterial doses (31.25/15.625/7.81 μg/ml), lending further credibility to the unanticipated nature of the quorum sensing inhibiting activity for the compounds of structure I for use in a method for the treatment of leaky gut, a method for the treatment of leaky gut syndrome, or a method for the improvement of at least one symptom associated with leaky gut syndrome according to the invention.

The results from these experiments indicate that compounds of structure I have a mechanism of action at sub-lethal antibacterial doses that reduces the pathogenicity of bacteria and hence shows an improvement in leaky gut syndrome. This effect is completely unanticipated; while the use of capsaicyns as TRPV1 agonists, an altogether unrelated mode of action, has been disclosed previously, the use of compounds of structure I against leaky gut, or to inhibit quorum sensing in other methods of treatment, has not been indicated.

The fact that the compounds inhibit quorum sensing at sub-lethal antibacterial doses is of particular importance. A lethal antibacterial effect is undesired, even possibly detrimental for a compound for the treatment of leaky gut, as such an effect undoubtedly would cause further imbalance in the gut microbiota, which likely would lead to worsening of the leaky gut. This is supported by the indications that antibiotics may worsen leaky gut syndrome.

Hence, in a method for the treatment of leaky gut, a method for the treatment of leaky gut syndrome, or a method for the improvement of at least one symptom associated with leaky gut syndrome according to the invention, dosages and dosage regimens are always chosen in order to avoid any lethal antibacterial effect, taking the half-life of the compound according to the invention into consideration when necessary. Lethal antibacterial effects are avoided by selecting a sufficiently low dose of compound I, as known by the skilled person and/or based on the findings of the experiment presented in Example 2 or similar experiments. This way, the gut microbiota, and thus its valuable effects such as nutrient absorption, is disturbed as little as possible.

A patient group of the present invention comprises subjects suffering from leaky gut and/or leaky gut syndrome, such as patients diagnosed with or suspected of having leaky gut or leaky gut syndrome. Another patient group of the present invention comprises subjects experiencing at least one symptom associated with leaky gut syndrome. In some embodiments, a subject is selected from at least one of these patient groups.

The subject may be a human or a non-human animal, such as a human or non-human mammal, preferably a human patient. The subject may be male or female. In some embodiments of the invention, the subject is an adult (i.e. 18 years of age or older). In certain embodiments, the subject is geriatric. In certain embodiments, the subject is not geriatric.

Leaky gut shares many of its symptoms with other health conditions. This can make the condition difficult to identify. The invention relates to both the treatment of leaky gut or of leaky gut syndrome as such and the improvement of at least one symptom associated with leaky gut syndrome in a subject. In some embodiments, the subject is selected from the group of subjects that experience one or more symptoms associated with leaky gut syndrome, and for which said one or more symptoms have not been attributed to any other clinical diagnosis, such as celiac disease, IBD, ulcerative colitis, or Crohn's disease. In other embodiments, the subject is selected from the group of subjects that experience one or more symptoms associated with leaky gut syndrome, and that further displays a significant deviation in the level of at least one biomarker associated with leaky gut, leaky gut syndrome and/or intestinal hyperpermeability compared to a normal level of the at least one biomarker. In specific embodiments, the latter group is further limited to subjects that have not been diagnosed with celiac disease, IBD, ulcerative colitis, or Crohn's disease.

One such biomarker is zonulin. Research and clinical studies of the protein zonulin and the zonulin signalling pathway demonstrate the clinical efficacy of zonulin as a biomarker of intestinal permeability. Zonulin has been found to increase permeability in the epithelial layer of the small intestine by reversibly modulating the intercellular tight junctions. Disregulation of the zonulin signalling pathway disrupts normal gut barrier function and alters immune responses. As a result, high levels of serum zonulin may point to the presence of increased intestinal permeability. Other relevant biomarkers include intestinal fatty acid binding protein (1-FABP), soluble CD14, interleukin-6 (IL-6), lipopolysaccharide (LPS).

The compounds for use in a method for the treatment of leaky gut according to the invention may be obtained commercially or using any procedure known to the person skilled in the art. Non-limiting examples of procedures for obtaining the compounds according to the invention are those disclosed by the applicant in EP 1670310 and in the Norwegian patent application NO 20200333.

In one aspect, the invention provides a compound of formula I, or a pharmaceutically acceptable salt or solvate thereof, for use in a method for the treatment of leaky gut.

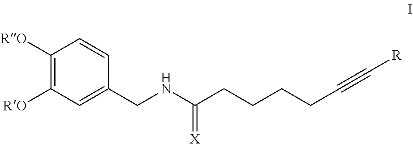

In a further aspect, the invention provides a compound of formula I, or a pharmaceutically acceptable salt or solvate thereof, for use in a method for the treatment of leaky gut syndrome.

In a further aspect, the invention provides a compound of formula I, or a pharmaceutically acceptable salt or solvate thereof, for use in a method for the improvement of at least one symptom associated with leaky gut syndrome in a subject, wherein the at least one symptom has not been attributed to any other clinical diagnosis.

In some embodiments, the subject does not suffer from celiac disease, IBD, ulcerative colitis, or Crohn's disease.

In a further aspect, the invention provides a compound of formula I, or a pharmaceutically acceptable salt or solvate thereof, for use in a method for the improvement of at least one symptom associated with leaky gut syndrome in a subject, wherein the subject displays a significant deviation in the level of at least one biomarker associated with leaky gut, leaky gut syndrome and/or intestinal hyperpermeability compared to a normal level of the at least one biomarker.

In some embodiments, the at least one biomarker is selected from the list of zonulin, I-FABP, soluble CD14, IL-6, LPS, and any combinations thereof. In specific embodiments, the at least one biomarker is zonulin.

In some embodiments, the normal level is the average level in the population. In some embodiments, the normal level is the average level for the subject. In some embodiments, the normal level is decided by a medical practitioner based on their common general knowledge.

In some embodiments, the subject does not suffer from celiac disease, IBD, ulcerative colitis, or Crohn's disease.

As discussed herein, there leaky gut and leaky gut syndrome are associated with increased permeability, such as hyperpermeability, of the gut. Hence, in another aspect, the invention provides a compound of formula I, for use in a method for the treatment of a increase in gut permeability in a subject who experiences one or more symptoms associated with leaky gut syndrome and who displays a significant deviation in the level of at least one biomarker associated with leaky gut, leaky gut syndrome and/or intestinal hyperpermeability compared to a normal level of the at least one biomarker.

The increase in gut permeability may be a pathological increase in gut permeability. Preferably, the increase in gut permeability is a subclinical increase in gut permeability.

In some embodiments, the invention provides a compound of formula I, for use in a method for the treatment of a subclinical increase in gut permeability in a subject who experiences one or more symptoms associated with leaky gut syndrome and who displays a significant deviation in the level of at least one biomarker associated with intestinal hyperpermeability compared to a normal level of the at least one biomarker.

Said biomarker may, e.g., be any of the biomarkers disclosed above, preferably zonulin.

In some embodiments, the subject has been diagnosed with leaky gut and/or leaky gut syndrome. In some embodiments, the subject has not been diagnosed with celiac disease, IBD, ulcerative colitis, or Crohn's disease.

For both the methods for the improvement of at least one symptom associated with leaky gut syndrome in a subject according to the invention the at least one symptom may be selected from the list of chronic diarrhoea, constipation, bloating, gas, stomach cramps, skin problems, food sensitivities, nutritional deficiencies; aches, pains, confusion, concentration difficulties, fatigue, chronic inflammation in any one or more parts of the body, and any combinations thereof. In some embodiments, the at least one symptom is selected from the list of chronic diarrhoea, constipation, bloating, gas, stomach cramps, acne, rashes, eczema, food sensitivities, nutritional deficiencies, joint pain, headaches, confusion, concentration difficulties, fatigue, chronic inflammation in any one or more parts of the body, and any combinations thereof. In some embodiments, the at least one symptom is selected from the list of chronic diarrhoea, constipation, bloating, gas, stomach cramps, chronic inflammation in any one or more parts of the body, and any combinations thereof.

In some embodiments, the at least one symptom is at least two symptoms selected from the list of chronic diarrhoea, constipation, bloating, gas, stomach cramps, acne, rashes, eczema, food sensitivities, nutritional deficiencies, joint pain, headaches, confusion, concentration difficulties, fatigue, chronic inflammation in any one or more parts of the body, and any combinations thereof. In some embodiments, the at least one symptom is at least three symptoms selected from the list of chronic diarrhoea, constipation, bloating, gas, stomach cramps, acne, rashes, eczema, food sensitivities, nutritional deficiencies, joint pain, headaches, confusion, concentration difficulties, fatigue, chronic inflammation in any one or more parts of the body, and any combinations thereof.

At least one symptom is improved by a significant amount, such as at least 5%, such as at least 10%, such as at least 20%, such as at least 50%, compared to without the use of the method according to the invention. The improvement may be evaluated by a medical practitioner using any method known in the art, or by using subject self-reported outcome measures.

In some embodiments, the invention provides a compound of formula I, or a pharmaceutically acceptable salt or solvate thereof, for use in a method for the treatment of leaky gut, wherein the subject of the treatment does not suffer from celiac disease, IBD, IBS, ulcerative colitis, or Crohn's disease.

In some embodiments, the invention provides a compound of formula I, or a pharmaceutically acceptable salt or solvate thereof, for use in a method for the treatment of leaky gut, wherein the treatment is prevention.

In some embodiments, the invention provides a compound of formula I, or a pharmaceutically acceptable salt or solvate thereof,
wherein
   X is selected from oxygen and sulphur,
   R is selected from the group comprising cyclohexyl, phenyl and substituted phenyl;
   wherein said substituted phenyl is substituted in any one or more positions with 1-5 identical or different substituents selected from the group comprising fluoro; chloro; bromo; iodo, cyano, nitro, trifluoromethyl, C₁-C₆ straight chain and branched alkoxy, C₁-C₆ sulfoxy, —S—C₁-C₆ alkyl, C₁-C₆ straight chain and branched alkyl, alkenyl, and alkynyl C₂-C₆ straight chain and branched alkenyl, C₂-C₆ straight chain and branched alkynyl, C₁-C₆ fluoroalkyl, chloroalkyl, bromoalkyl, and iodoalkyl, COO—C₁-C₆ alkyl, O(CO)—C₁-C₆ alkyl, NH—C₁-C₆ alkyl, N(C₁-C₆ alkyl)₂, CON(C₁-C₆ alkyl)₂, and NH(CO)—C₁-C₆ alkyl;
   R′ is selected from the group comprising hydrogen, C₁-C₆ straight chain and branched alkyl, and C₃-C₆ cycloalkyl, and
   R″ is selected from the group comprising hydrogen, benzyl, and acetyl, for use in a method for the treatment of leaky gut.

In some embodiments, the invention provides a compound of formula I, or a pharmaceutically acceptable salt or solvate thereof, for use in a method for the treatment of leaky gut, a method for the treatment of leaky gut syndrome, or a method for the improvement of at least one symptom associated with leaky gut syndrome, wherein
X is selected from oxygen and sulphur,
R is selected from the group comprising phenyl and substituted phenyl;
wherein said substituted phenyl is substituted in any one or more positions with 1-5 identical or different substituents selected from the group comprising fluoro; chloro; bromo; iodo, cyano, nitro, trifluoromethyl, C₁-C₆ straight chain and branched alkoxy, C₁-C₆ sulfoxy, —S—C₁-C₆ alkyl, C₁-C₆ straight chain and branched alkyl, alkenyl, and alkynyl C₂-C₆ straight chain and branched alkenyl, C₂-C₆ straight chain and branched alkynyl, C₁-C₆ fluoroalkyl, chloroalkyl, bromoalkyl, and iodoalkyl, COO—C₁-C₆ alkyl, O(CO)—C₁-C₆ alkyl, NH—C₁-C₆ alkyl, N(C₁-C₆ alkyl)₂, CON(C₁-C₆ alkyl)₂, and NH(CO)—C₁-C₆ alkyl;
R′ is selected from the group comprising hydrogen, C₁-C₆ straight chain and branched alkyl, and C₃-C₆ cycloalkyl, and
R″ is selected from the group comprising hydrogen, benzyl, and acetyl.

R may be selected from the group comprising phenyl and substituted phenyl, wherein said substituted phenyl is substituted in any one or more positions with 1-5 identical or different substituents selected from the group comprising fluoro; chloro; bromo; iodo; cyano; nitro; trifluoromethyl; C₁-C₆ straight chain and branched alkoxy; C₁-C₆ sulfoxy; —S—C₁-C₆ alkyl; C₁-C₆ straight chain and branched alkyl, alkenyl, and alkynyl; C₁-C₆ fluoroalkyl, chloroalkyl, bromoalkyl, and iodoalkyl; COO—C₁-C₆ alkyl, O(CO)—C₁-C₆ alkyl, NH—C₁-C₆ alkyl, N(C₁-C₆ alkyl)₂, CON(C₁-C₆ alkyl)₂, and NH(CO)—C₁-C₆ alkyl. Such variants are denoted a1.

R may be selected from the group comprising phenyl and substituted phenyl, wherein said substituted phenyl is substituted in any one or more positions with 1-5 identical or different substituents selected from the group comprising fluoro; chloro; bromo; iodo; cyano; nitro; trifluoromethyl; C₁-C₆ straight chain and branched alkoxy; C₁-C₆ sulfoxy; —S—C₁-C₆ alkyl; C₁-C₆ straight chain and branched alkyl, alkenyl, and alkynyl; and C₁-C₆ fluoroalkyl, chloroalkyl, bromoalkyl, and iodoalkyl. Such variants are denoted a2.

R may be selected from the group comprising phenyl and substituted phenyl, wherein said substituted phenyl is substituted in any one or more positions with 1-5 identical or different substituents selected from the group comprising fluoro; chloro; bromo; iodo; cyano; nitro; trifluoromethyl, NH—C₁-C₆ alkyl, N(C₁-C₆ alkyl)₂, COO—C₁-C₆ alkyl, and O(CO)—C₁-C₆ alkyl. Such variants are denoted a3.

R may be selected from the group comprising phenyl and substituted phenyl, wherein said substituted phenyl is substituted in any one or more positions with 1-5 identical or different substituents selected from the group comprising fluoro; chloro; bromo; iodo; C₁-C₆ straight chain and branched alkyl, alkenyl, and alkynyl; NH—C₁-C₆ alkyl, N(C₁-C₆ alkyl)₂, and C₁-C₆ straight chain and branched alkoxy. Such variants are denoted a4.

R may be selected from the group comprising phenyl and substituted phenyl, wherein said substituted phenyl is substituted in any one or more positions with 1-5 identical or different substituents selected from the group comprising fluoro; chloro; bromo; iodo; C₁-C₃ straight chain and branched alkyl, alkenyl, and alkynyl; COO—C₁-C₃ alkyl, and O(CO)—C₁-C₃ alkyl. Such variants are denoted a5.

R may be selected from the group comprising phenyl and substituted phenyl, wherein said substituted phenyl is substituted in any one or more positions with 1-5 identical or different substituents selected from the group comprising C₁-C₆ straight chain and branched alkyl, alkenyl, and alkynyl; and C₁-C₆ fluoroalkyl, chloroalkyl, bromoalkyl, and iodoalkyl. Such variants are denoted a6.

R may be phenyl. Such variants are denoted a7.

Said substituted phenyl may have one substituent, such as in 2-position, such as in 3-position, such as in 4-position. Said phenyl ring may have two substituents, such as in positions 2 and 6, such as in positions 2 and 5, such as in positions 2 and 3, such as in positions 3 and 5, such as in positions 2 and 4, such as in positions 3 and 4. Said phenyl ring may have three substituents, such as in positions 2, 3, and 6, such as in positions 2, 4, and 6, such as in positions 2, 3, and 4, such as in positions 2, 3, and 5, such as in positions 3, 4, and 5. Said phenyl ring may have four substituents, such as in positions 2, 3, 4, and 6, such as in positions 2, 3, 4, and 5, such as in positions 2, 3, 5, and 6. Said phenyl ring may have five substituents.

In all variants a1-a7, the group from which R is selected may further comprise cyclohexyl.

In some variants, two of said substituents on the substituted phenyl are identical to each other. In some variants, three of the substituents are identical to each other. In some variants, four of the substituents are identical to each other. In some variants, five of the substituents are identical to each other. In other variants, all of said substituents are different from each other.

R′ may be selected from the group comprising hydrogen, C₁-C₆ straight chain and branched alkyl, and C₃-C₆ cycloalkyl. Such variants are denoted b1.

R′ may be selected from the group comprising C₃-C₆ straight chain and branched alkyl, and C₃-C₆ cycloalkyl. Such variants are denoted b2.

R′ may be selected from the group comprising hydrogen and C₁-C₃ straight chain and branched alkyl. Such variants are denoted b3.

R′ may be selected from methyl and ethyl. Such variants are denoted b4.

R″ may be selected from the group comprising hydrogen, benzyl, and acetyl. Such variants are denoted c1.

R″ may be hydrogen. Such variants are denoted c2.

X may be oxygen or sulphur. Such variants are denoted d1.

X may be oxygen. Such variants are denoted d2.

X may be sulphur. Such variants are denoted d3.

It is to be understood that each selection of each possible X, R group, R′ group, and R″ group disclosed herein is to be interpreted as being disclosed for use in any combination with one or more of each and every other election of possible X, R group, R′ group and R″ group disclosed herein.

Thus, in some embodiments, the invention provides compound of formula I, or a pharmaceutically acceptable salt or solvate thereof, for use in a method for the treatment of leaky gut, wherein the selections of X, R, R′, and R″ are as listed below: a1+b1+c1+d1, a1+b2+c1+d1, a1+b3+c1+d1, a1+b4+c1+d1, a2+b1+c1+d1, a2+b2+c1+d1, a2+b3+c1+d1, a2+b4+c1+d1, a3+b1+c1+d1, a3+b2+c1+d1, a3+b3+c1+d1, a3+b4+c1+d1, a4+b1+c1+d1, a4+b2+c1+d1, a4+b3+c1+d1, a4+b4+c1+d1, a5+b1+c1+d1, a5+b2+c1+d1, a5+b3+c1+d1, a5+b4+c1+d1, a6+b1+c1+d1, a6+b2+c1+d1, a6+b3+c1+d1, a6+b4+c1+d1, a7+b1+c1+d1, a7+b2+c1+d1, a7+b3+c1+d1, a7+b4+c1+d1, a1+b1+c2+d1, a1+b2+c2+d1, a1+b3+c2+d1, a1+b4+c2+d1, a2+b1+c2+d1, a2+b2+c2+d1, a2+b3+c2+d1, a2+b4+c2+d1, a3+b1+c2+d1, a3+b2+c2+d1, a3+b3+c2+d1, a3+b4+c2+d1, a4+b1+c2+d1, a4+b2+c2+d1, a4+b3+c2+d1, a4+b4+c2+d1, a5+b1+c2+d1, a5+b2+c2+d1, a5+b3+c2+d1, a5+b4+c2+d1, a6+b1+c2+d1, a6+b2+c2+d1, a6+b3+c2+d1, a6+b4+c2+d1, a7+b1+c2+d1, a7+b2+c2+d1, a7+b3+c2+d1, a7+b4+c2+d1, a1+b1+c1+d2, a1+b2+c1+d2, a1+b3+c1+d2, a1+b4+c1+d2, a2+b1+c1+d2, a2+b2+c1+d2, a2+b3+c1+d2, a2+b4+c1+d2, a3+b1+c1+d2, a3+b2+c1+d2, a3+b3+c1+d2, a3+b4+c1+d2, a4+b1+c1+d2, a4+b2+c1+d2, a4+b3+c1+d2, a4+b4+c1+d2, a5+b1+c1+d2, a5+b2+c1+d2, a5+b3+c1+d2, a5+b4+c1+d2, a6+b1+c1+d2, a6+b2+c1+d2, a6+b3+c1+d2, a6+b4+c1+d2, a7+b1+c1+d2, a7+b2+c1+d2, a7+b3+c1+d2, a7+b4+c1+d2, a1+b1+c2+d2, a1+b2+c2+d2, a1+b3+c2+d2, a1+b4+c2+d2, a2+b1+c2+d2, a2+b2+c2+d2, a2+b3+c2+d2, a2+b4+c2+d2, a3+b1+c2+d2, a3+b2+c2+d2, a3+b3+c2+d2, a3+b4+c2+d2, a4+b1+c2+d2, a4+b2+c2+d2, a4+b3+c2+d2, a4+b4+c2+d2, a5+b1+c2+d2, a5+b2+c2+d2, a5+b3+c2+d2, a5+b4+c2+d2, a6+b1+c2+d2, a6+b2+c2+d2, a6+b3+c2+d2, a6+b4+c2+d2, a7+b1+c2+d2, a7+b2+c2+d2, a7+b3+c2+d2, a7+b4+c2+d2, a1+b1+c1+d3, a1+b2+c1+d3, a1+b3+c1+d3, a1+b4+c1+d3, a2+b1+c1+d3, a2+b2+c1+d3, a2+b3+c1+d3, a2+b4+c1+d3, a3+b1+c1+d3, a3+b2+c1+d3, a3+b3+c1+d3, a3+b4+c1+d3, a4+b1+c1+d3, a4+b2+c1+d3, a4+b3+c1+d3, a4+b4+c1+d3, a5+b1+c1+d3, a5+b2+c1+d3, a5+b3+c1+d3, a5+b4+c1+d3, a6+b1+c1+d3, a6+b2+c1+d3, a6+b3+c1+d3, a6+b4+c1+d3, a7+b1+c1+d3, a7+b2+c1+d3, a7+b3+c1+d3, a7+b4+c1+d3, a1+b1+c2+d3, a1+b2+c2+d3, a1+b3+c2+d3, a1+b4+c2+d3, a2+b1+c2+d3, a2+b2+c2+d3, a2+b3+c2+d3, a2+b4+c2+d3, a3+b1+c2+d3, a3+b2+c2+d3, a3+b3+c2+d3, a3+b4+c2+d3, a4+b1+c2+d3, a4+b2+c2+d3, a4+b3+c2+d3, a4+b4+c2+d3, a5+b1+c2+d3, a5+b2+c2+d3, a5+b3+c2+d3, a5+b4+c2+d3, a6+b1+c2+d3, a6+b2+c2+d3, a6+b3+c2+d3, a6+b4+c2+d3, a7+b1+c2+d3, a7+b2+c2+d3, a7+b3+c2+d3, a7+b4+c2+d3.

The letter-number combinations refer to the variants defined above, so that e.g. a1+b1+c1+d1 denotes a compound wherein R is selected from the group comprising phenyl and substituted phenyl, wherein said substituted phenyl is substituted in any one or more positions with 1-5 identical or different substituents selected from the group comprising fluoro; chloro; bromo; iodo; cyano; nitro; trifluoromethyl; C₁-C₆ straight chain and branched alkoxy; C₁-C₆ sulfoxy; —S—C₁-C₆ alkyl; C₁-C₆ straight chain and branched alkyl, alkenyl, and alkynyl; C₁-C₆ fluoroalkyl, chloroalkyl, bromoalkyl, and iodoalkyl; COO—C₁-C₆ alkyl, O(CO)—C₁-C₆ alkyl, NH—C₁-C₆ alkyl, N(C₁-C₆ alkyl)₂, CON(C₁-C₆ alkyl)₂, and NH(CO)—C₁-C₆ alkyl (a1), R′ is selected from the group comprising hydrogen, C₁-C₆ straight chain and branched alkyl, and C₃-C₆ cycloalkyl (b1), R″ is selected from the group comprising hydrogen, benzyl, and acetyl (c1), and X is selected from oxygen and sulphur (d1), and so on.

In some embodiments, the invention provides a compound of formula I, or a pharmaceutically acceptable salt or solvate thereof, for use in a method for the treatment of leaky gut, a method for the treatment of leaky gut syndrome, or a method for the improvement of at least one symptom associated with leaky gut syndrome, wherein said compound is a capsaicyn. In some embodiments, the invention provides a compound of formula I, or a pharmaceutically acceptable salt or solvate thereof, for use in a method for the treatment of leaky gut, a method for the treatment of leaky gut syndrome, or a method for the improvement of at least one symptom associated with leaky gut syndrome, wherein said compound is a thioamide capsaicyn.

In preferred embodiments, the invention provides a compound of formula I, or a pharmaceutically acceptable salt or solvate thereof, for use in a method for the treatment of leaky gut, a method for the treatment of leaky gut syndrome, or a method for the improvement of at least one symptom associated with leaky gut syndrome, wherein R is a substituted phenyl that has at least one substituent in the 4-position, said at least one substituent being selected from a group listed above for one of alternatives a1, a2, a3, a4, a5, or a6.

In other preferred embodiments, the invention provides a compound of formula I, or a pharmaceutically acceptable salt or solvate thereof, for use in a method for the treatment of leaky gut, a method for the treatment of leaky gut syndrome, or a method for the improvement of at least one symptom associated with leaky gut syndrome, wherein R is a substituted phenyl that has only one substituent, said substituent being in the 4-position and being selected from a group listed above for one of alternatives a1, a2, a3, a4, a5, or a6.

In preferred embodiments, the invention provides a compound of formula I, or a pharmaceutically acceptable salt or solvate thereof, for use in a method for the treatment of leaky gut, a method for the treatment of leaky gut syndrome, or a method for the improvement of at least one symptom associated with leaky gut syndrome, wherein X is oxygen; R is phenyl or substituted phenyl as defined above (a1, a2, a3, a4, a5, a6, or a7); R′ is methyl, ethyl, or isopropyl; and R″ is hydrogen, acetyl or benzyl.

In other preferred embodiments, the invention provides a compound of formula I, or a pharmaceutically acceptable salt or solvate thereof, for use in a method for the treatment of leaky gut, a method for the treatment of leaky gut syndrome, or a method for the improvement of at least one symptom associated with leaky gut syndrome, wherein X is oxygen; R is phenyl, 4-methylphenyl, 4-chlorophenyl, or 4-acetoxyphenyl; R′ is methyl, ethyl, or isopropyl; and R″ is hydrogen, acetyl or benzyl.

Preferred, non-limiting examples of compounds for use in a method for the treatment of leaky gut, a method for the treatment of leaky gut syndrome, or a method for the improvement of at least one symptom associated with leaky gut syndrome according to the invention include the list of compounds of formula I wherein R, R′, R″, and X are selected as follows:
R=phenyl; R′=methyl, R″=hydrogen, X=oxygen (phenylcapsaicin, V)
R=phenyl; R′=methyl, R″=acetyl, X=oxygen
R=phenyl; R′=methyl, R″=benzyl, X=oxygen
R=phenyl; R′=ethyl, R″=acetyl, X=oxygen
R=4-methylphenyl; R′=methyl, R″=acetyl, X=oxygen
R=4-chlorophenyl; R′=methyl, R″=acetyl, X=oxygen
R=4-methylphenyl; R′=methyl, R″=hydrogen, X=oxygen
R=4-acetoxyphenyl; R′=methyl, R″=hydrogen, X=oxygen
R=4-methylphenyl; R′=methyl, R″=benzyl, X=oxygen
R=4-acetoxyphenyl; R′=ethyl, R″=benzyl, X=oxygen
R=cyclohexyl; R′=methyl, R″=hydrogen, X=oxygen
R=cyclohexyl; R′=ethyl, R″=hydrogen, X=oxygen

In some embodiments, the invention provides a compound of formula I, or a pharmaceutically acceptable salt or solvate thereof, for use in a method for the treatment of leaky gut, a method for the treatment of leaky gut syndrome, or a method for the improvement of at least one symptom associated with leaky gut syndrome, wherein the compound is selected from the group of phenyl-substituted 6-yne derivatives of capsaicin, often referred to as phenylcapsaicins, wherein R is phenyl or substituted phenyl according to any of variants a1-a7 above.

In some embodiments, the invention provides a compound of formula II, or a pharmaceutically acceptable salt or solvate thereof, for use in a method for the treatment of leaky gut, a method for the treatment of leaky gut syndrome, or a method for the improvement of at least one symptom associated with leaky gut syndrome, wherein R denotes a phenyl or substituted phenyl selected from the variants a1-a7 above.

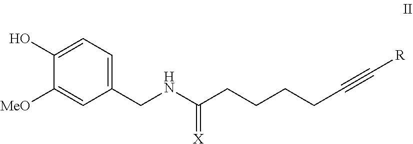

In some embodiments, the invention provides a compound of formula II, or a pharmaceutically acceptable salt or solvate thereof, for use in a method for the treatment of leaky gut, a method for the treatment of leaky gut syndrome, or a method for the improvement of at least one symptom associated with leaky gut syndrome, wherein R denotes cyclohexyl or a phenyl or substituted phenyl selected from the variants a1-a7 above, and wherein X is oxygen.

In some embodiments, the invention provides a compound of formula II, or a pharmaceutically acceptable salt or solvate thereof, for use in a method for the treatment of leaky gut, a method for the treatment of leaky gut syndrome, or a method for the improvement of at least one symptom associated with leaky gut syndrome, wherein R denotes a phenyl or substituted phenyl selected from the variants a1-a7 above, and wherein X is oxygen.

In some embodiments, the invention provides a compound of formula II, or a pharmaceutically acceptable salt or solvate thereof, for use in a method for the treatment of leaky gut, a method for the treatment of leaky gut syndrome, or a method for the improvement of at least one symptom associated with leaky gut syndrome, wherein R denotes a phenyl or substituted phenyl selected from the variants a1-a7 above, and wherein X is sulphur.

In some embodiments, the invention provides a compound of formula III, or a pharmaceutically acceptable salt or solvate thereof, for use in a method for the treatment of leaky gut, a method for the treatment of leaky gut syndrome, or a method for the improvement of at least one symptom associated with leaky gut syndrome, wherein X is selected from oxygen and sulphur (variant d1).

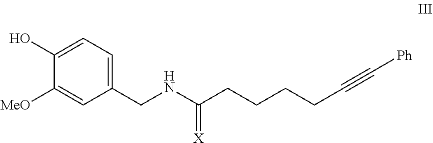

In some embodiments, the invention provides a compound of formula IV, or a pharmaceutically acceptable salt or solvate thereof, for use in a method for the treatment of leaky gut, a method for the treatment of leaky gut syndrome, or a method for the improvement of at least one symptom associated with leaky gut syndrome.

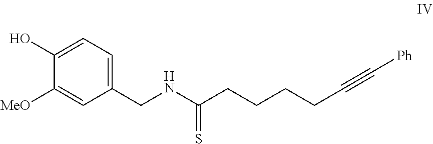 [SMILES: COc1cc(CNC(=S)CCCCC#Cc2ccccc2)ccc1O]

In some embodiments, the invention provides phenylcapsaicin of formula V, or a pharmaceutically acceptable salt or solvate thereof, for use in a method for the treatment of leaky gut, a method for the treatment of leaky gut syndrome, or a method for the improvement of at least one symptom associated with leaky gut syndrome.

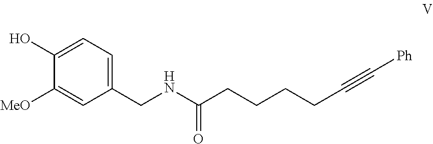 [SMILES: COc1cc(CNC(=O)CCCCC#Cc2ccccc2)ccc1O]

Alternatively, R′, R″, and X may be selected from any combination of groups outlined above (b1+c1+d1, b1+c2+d1, b1+c1+d2, b1+c2+d2, b1+c1+d3, b1+c2+d3, b2+c1+d1, b2+c2+d1, b2+c1+d2, b2+c2+d2, b2+c1+d3, b2+c2+d3, b3+c1+d1, b3+c2+d1, b3+c1+d2, b3+c2+d2, b3+c1+d3, b3+c2+d3, b4+c1+d1, b4+c2+d1, b4+c1+d2, b4+c2+d2, b4+c1+d3, b4+c2+d3), while R is selected from C₁-C₆ straight chained or branched alkyl, such as tert-butyl, such as cyclohexyl, such as cyclohexenyl, such as cyclohexadienyl.

The compounds of the invention may contain one or more chiral centres and/or double bonds, and may therefore exist in different stereoisomeric forms, such as double-bond isomers (i.e., geometric isomers), enantiomers, and/or diastereomers. It is to be understood that both stereomerically pure forms (e.g., geometrically pure, enantiomerically pure, or diastereomerically pure) and stereoisomeric mixtures are encompassed in the invention. The invention is considered to extend to diastereomers and enantiomers, as well as racemic mixtures. Compounds herein described may be resolved into their geometric isomers, enantiomers and/or diastereomers, using methods known in the art.

Any of the compounds according to the invention may be provided in the form of a prodrug. The term “prodrug” refers to a derivative of a pharmacologically active compound that, after administration, undergoes a transformation, such as being metabolised within the body, to release the pharmacologically active drug. A prodrug may, but need not necessarily, be pharmacologically inactive until converted into the active compound. A prodrug may be obtained by derivatising one or more functional groups in the active compound with a progroup, i.e. a group that masks a functional group within the active compound and that undergoes a transformation under the specified conditions of use, such as in vivo, to release said functional group. The progroup should be nontoxic. A wide range of progroups, and methods for providing prodrugs, are known to the person skilled in the art.

The compounds for use in a method for the treatment of leaky gut, a method for the treatment of leaky gut syndrome, or a method for the improvement of at least one symptom associated with leaky gut syndrome according to the invention may be present as an active ingredient in a desired dosage unit formulation, such as a pharmaceutically acceptable composition containing conventional pharmaceutically acceptable carriers.

Compositions for use in a method for the treatment of leaky gut, a method for the treatment of leaky gut syndrome, or a method for the improvement of at least one symptom associated with leaky gut syndrome according to the invention are prepared from the compounds of structure I according to the invention in substantially pure form. In some embodiments, the purity of the compound of structure I used to formulate the composition is at least about 95%, such as at least 96%, 97%, 98%, or 99%. Preferably, the purity of the compound is at least 98%.

The composition may further include one or more of any conventional, pharmaceutically acceptable excipients and/or carriers, e.g. solvents, fillers, diluents, binders, lubricants, glidants, viscosity modifiers, surfactants, dispersing agents, disintegration agents, emulsifying agents, wetting agents, suspending agents, thickeners, buffers, pH modifiers, absorption-delaying agents, stabilisers, antioxidants, preservatives, antifungal agents, chelating agents, adjuvants, sweeteners, aromas, and colouring agents. Conventional formulation techniques known in the art, e.g., conventional mixing, dissolving, suspending, granulating, drageemaking, levigating, emulsifying, encapsulating, entrapping or compressing processes, may be used to formulate the composition.

In some embodiments, the composition for use in a method for the treatment of leaky gut, a method for the treatment of leaky gut syndrome, or a method for the improvement of at least one symptom associated with leaky gut syndrome is formulated for oral, and/or rectal, and/or intraperitoneal administration.

The amount of the compound of structure I according to the invention present in the composition can vary. In some embodiments, the amount of the compound according to the invention present in the composition is 1-50% by weight, such as 1-30%, such as 20-50%. In other embodiments, the amount of the compound according to the invention present in the composition is 30-70% by weight, such as 40-60%. In yet other embodiments, the amount of the compounds according to the invention present in the composition is 50-100% by weight, such as 50-70%, such as 50-80%, such as 60-98%, such as 70-95%, such as 80-99%, such as 95-100%.

Further, the composition for use in a method for the treatment of leaky gut, a method for the treatment of leaky gut syndrome, or a method for the improvement of at least one symptom associated with leaky gut syndrome according to the invention is substantially free of contaminants or impurities. In some embodiments, the level of contaminants or impurities other than residual solvent in the composition is below about 5% relative to the combined weight of the compounds according to the invention and the intended other ingredients. In certain embodiments, the level of contaminants or impurities other than residual solvent in the composition is no more than about 2% or 1% relative to the combined weight of the compounds according to the invention and the intended other ingredients.

In certain embodiments, the compound or composition for use in a method for the treatment of leaky gut, a method for the treatment of leaky gut syndrome, or a method for the improvement of at least one symptom associated with leaky gut syndrome according to the invention is sterile. Sterilisation can be achieved by any suitable method, including but not limited to by applying heat, chemicals, irradiation, high pressure, filtration, or combinations thereof.

In certain embodiments, the compound or composition for use in a method for the treatment of leaky gut, a method for the treatment of leaky gut syndrome, or a method for the improvement of at least one symptom associated with leaky gut syndrome according to the invention is formulated as a medicament, an active pharmaceutical ingredient (API), a food additive, a food supplement, a dietary supplement, a nutritional supplement, an OTC supplement, a medical food, and/or a pharmaceutical grade supplement.

In certain embodiments, the method according to the invention comprises the step of administering to a subject a compound or composition according to the invention, wherein the compound or composition is formulated as a medicament, an active pharmaceutical ingredient (API), a food additive, a food supplement, a dietary supplement, a nutritional supplement, an OTC supplement, a medical food, and/or a pharmaceutical grade supplement.

The compound or composition for use in a method for the treatment of leaky gut, a method for the treatment of leaky gut syndrome, or a method for the improvement of at least one symptom associated with leaky gut syndrome according to the invention will be administered to a subject in a therapeutically effective dose, wherein the compound or composition has no or sublethal antibacterial effect, such as on Proteobacteria, Actinobacteria, Firmicutes, Bacteroidetes, Bacteroides, Porphyromonas, Bifidobacterium, Lactobacillus, Clostridium, Prevotella, Ruminococcus, Alistipes, Dorea, Eubacterium, Faecalibacterium, Collinsella, Roseburia, Coprococcus, Holdemania.

Appropriate dosages may depend on the compound to be used, the stage of the condition, age and weight of the patient, etc. and may be routinely determined by the skilled practitioner according to principles well known in the art. A suitable daily dosage of the compound according to the invention may range from about 0.001 mg/kg body weight to 1.0 mg/kg body weight. In some embodiments, the range is limited by the requirement that the dosage should not have a lethal antibacterial effect. For example, in some embodiments, the daily dose may be 0.001-00.1 mg/kg body weight, such as 0.001-0.005 mg/kg body weight such as 0.003-0.008 mg/kg body weight, such as 0.005-0.01 mg/kg body weight. In other embodiments, the daily dose may be 0.005-0.05 mg/kg body weight, such as 0.005-0.02 mg/kg body weight, such as 0.008-0.05 mg/kg body weight. In other embodiments, the daily dose may be 0.01-0.1 mg/kg body weight, such as 0.01-0.05 mg/kg body weight such as 0.03-0.08 mg/kg body weight, such as 0.05-0.1 mg/kg body weight. In other embodiments, the daily dose may be 0.05-0.5 mg/kg body weight, such as 0.05-0.2 mg/kg body weight, such as 0.08-0.5 mg/kg body weight. In yet other embodiments, the daily dose may be 0.1-1.0 mg/kg body weight, such as 0.3-0.08 mg/kg body weight, such as 0.5-1.0 mg/kg body weight.

The therapeutically effective dose of the compound according to the invention can be administered in a single dose or in divided doses. The compound or composition according to the invention can be administered once, twice or more times a day, once every two days, once every three days, twice a week or once a week, or as deemed appropriate by a medical professional. In certain embodiments, the compound or composition according to the invention is administered once daily. In other embodiments, the compound or composition according to the invention is administered twice daily. In some embodiments, the dosage regimen is predetermined and the same for the entire patient group. In other embodiments, the dosage and the frequency of administration of treatment with the compound or composition according to the invention is determined by a medical professional, based on factors including, but not limited to, the stage of the disease, the severity of symptoms, the route of administration, the age, body weight, general health, gender and/or diet of the subject, and/or the response of the subject to the treatment. When selecting a dosage regimen, care should be taken to avoid lethal antibacterial effects.

In some embodiments, the therapeutically effective dose is administered at regular intervals. In other embodiments, the dose is administered when needed or sporadically.

The compound or composition according to the invention may be administered by a medical professional or by self-administration. The compound or composition according to the invention may, depending on factors such as formulation and route of administration, be administered with food or without food. In some embodiments, the compound or composition according to the invention is administered at specific times of day.

The compound or composition for use in a method for the treatment of leaky gut, a method for the treatment of leaky gut syndrome, or a method for the improvement of at least one symptom associated with leaky gut syndrome according to the invention may be administered locally or systemically. The compound or composition according to the invention may be administered by any administration route, including but not limited to, orally, intraperitoneally, sublingually, buccally, rectally, and enterally.

In some embodiments, the compound or composition is administered orally, and/or rectally, and/or intraperitoneally.

In preferred embodiments, the compound or composition is administered orally. In certain embodiments, the compound or composition is administered with a meal or before a meal. In certain embodiments, the compound or composition is administered at a non-antibacterial dose in a formulation that maximises time in the gut and lowers the metabolism rate of the drug.

In some embodiments, the compound or composition for use in a method for the treatment of leaky gut, a method for the treatment of leaky gut syndrome, or a method for the improvement of at least one symptom associated with leaky gut syndrome according to the invention is administered interperitoneally, such as by interperitoneal injection.

Preferred unit dosage formulations are those containing a therapeutically effective dose, as hereinbefore recited, or an appropriate fraction thereof, of a compound of structure I for use in a method for the treatment of leaky gut, a method for the treatment of leaky gut syndrome, or a method for the improvement of at least one symptom associated with leaky gut syndrome according to the invention. A composition for use in a method for the treatment of leaky gut, a method for the treatment of leaky gut syndrome, or a method for the improvement of at least one symptom associated with leaky gut syndrome may be presented in unit dosage form as a single dose wherein all active and inactive ingredients are combined in a suitable system and components do not need to be mixed before administration. Alternatively, a composition may be presented as a kit in which the drug, excipients and carriers are provided in two or more separate containers (e.g., ampules, vials, tubes, bottles or syringes) and need to be combined to form the composition to be administered. A kit may contain one or more compounds according to the invention or a composition according to the invention and all other ingredients in unit dosage form, or in two or more separate containers, and may contain instructions for storing, preparing, administering and/or using the composition.

In some embodiments, the duration of the use of the compound or composition for use in a method for the treatment of leaky gut, a method for the treatment of leaky gut syndrome, or a method for the improvement of at least one symptom associated with leaky gut syndrome according to the invention is determined by the formulation used and/or by the underlying mechanism of action detected and/or suspected in a subject to be treated and/or by the specific symptoms experienced by a subject to be treated. In some embodiments, treatment is sustained until no further improvement can be expected based on the symptoms of the subject that is treated. In certain embodiments, the duration of the treatment with the compound or composition according to the invention is at least three days, at least one week, at least two weeks, at least one month, at least three months, such as three months, six months, nine months. In some embodiments, the treatment is stopped when the symptoms associated with leaky gut abate. In some embodiments, the duration is determined by a medical professional, based on factors including but not limited to the nature and severity of the symptoms, the route of administration, the age, body weight, general health, gender and/or diet of the subject, and/or the response of the subject to the treatment. In some embodiments, treatment is repeated upon recurrence of symptoms associated with leaky gut. In other embodiments, the compound or composition is administered chronically, such as in a continuous mode as opposed to an acute mode, so as to maintain the initial therapeutic effect for an extended period of time.

In certain embodiments, the compound or composition according to the invention is administered alone. In other embodiments, the compound or composition according to the invention is administered in combination with one or more other therapeutic agents. Said one or more other therapeutic agents may be known to have an effect against leaky gut and/or may have an additive or synergistic mechanism of action on leaky gut treatment together with the compound or composition of the invention. Said one or more other therapeutic agents may be known to have an effect against other diseases or conditions associated with and/or present at the same time as leaky gut and/or may have an additive or synergistic mechanism of action on leaky gut treatment together with the compound or composition of the invention. In some embodiments, the compound or composition according to the invention is administered as part of a combination therapy. Combination therapies comprising a compound or composition according to the invention may refer to compositions that comprise the compound or composition according to the invention in combination with one or more therapeutic agents, and/or co-administration of the compound or composition according to the invention with one or more therapeutic agents wherein the compound or composition according to the invention and the other therapeutic agent or agents have not been formulated in the same composition. When using separate formulations, the compound or composition according to the invention may be administered simultaneously, intermittent, staggered, prior to, subsequent to, or combinations of these, with the administration of another therapeutic agent. When using combination therapies, care should be taken to avoid lethal antibacterial effects.

The embodiments and features described in the context of one aspect, e.g. for the aspect directed to the compound or composition for use in a method for the treatment of leaky gut, a method for the treatment of leaky gut syndrome, or a method for the improvement of at least one symptom associated with leaky gut syndrome, also apply to the other aspects of the invention.

In a further aspect, the invention provides a method for the treatment of leaky gut, a method for the treatment of leaky gut syndrome, or a method for the improvement of at least one symptom associated with leaky gut syndrome, the method comprising the step of administering an effective amount of a compound of formula I, or any pharmaceutically acceptable salt or solvate thereof, to a subject.

In some embodiments, the invention provides a method for the treatment of leaky gut, a method for the treatment of leaky gut syndrome, or a method for the improvement of at least one symptom associated with leaky gut syndrome, the method comprising the step of administering a composition comprising an effective amount of a compound of formula I, or any pharmaceutically acceptable salt or solvate thereof, to a subject.

In some embodiments, the invention provides a method for the treatment of leaky gut, leaky gut syndrome, or increased intestinal permeability, the method comprising the step of administering to a subject an effective amount of a compound of formula I, or a pharmaceutically acceptable salt or solvate thereof,
wherein
   X is selected from oxygen and sulphur,
   R is selected from the group comprising cyclohexyl, phenyl and substituted phenyl;
   wherein said substituted phenyl is substituted in any one or more positions with 1-5 identical or different substituents selected from the group comprising fluoro; chloro; bromo; iodo, cyano, nitro, trifluoromethyl, C₁-C₆ straight chain and branched alkoxy, C₁-C₆ sulfoxy, —S—C₁-C₆ alkyl, C₁-C₆ straight chain and branched alkyl, alkenyl, and alkynyl C₂-C₆ straight chain and branched alkenyl, C₂-C₆ straight chain and branched alkynyl, C₁-C₆ fluoroalkyl, chloroalkyl, bromoalkyl, and iodoalkyl, COO—C₁-C₆ alkyl, O(CO)—C₁-C₆ alkyl, NH—C₁-C₆ alkyl, N(C₁-C₆ alkyl)₂, CON(C₁-C₆ alkyl)₂, and NH(CO)—C₁-C₆ alkyl;
R′ is selected from the group comprising hydrogen, C₁-C₆ straight chain and branched alkyl, and C₃-C₆ cycloalkyl, and
R″ is selected from the group comprising hydrogen, benzyl, and acetyl, or a composition comprising said compound of formula I.

In some embodiments, the invention provides a method for the treatment of leaky gut, leaky gut syndrome, or increased intestinal permeability, the method comprising the step of administering to a subject an effective amount of a compound of formula I, or a pharmaceutically acceptable salt or solvate thereof,
wherein
   X is selected from oxygen and sulphur,
   R is selected from the group comprising phenyl and substituted phenyl;
   wherein said substituted phenyl is substituted in any one or more positions with 1-5 identical or different substituents selected from the group comprising fluoro; chloro; bromo; iodo, cyano, nitro, trifluoromethyl, C₁-C₆ straight chain and branched alkoxy, C₁-C₆ sulfoxy, —S—C₁-C₆ alkyl, C₁-C₆ straight chain and branched alkyl, alkenyl, and alkynyl C₂-C₆ straight chain and branched alkenyl, C₂-C₆ straight chain and branched alkynyl, C₁-C₆ fluoroalkyl, chloroalkyl, bromoalkyl, and iodoalkyl, COO—C₁-C₆ alkyl, O(CO)—C₁-C₆ alkyl, NH—C₁-C₆ alkyl, N(C₁-C₆ alkyl)₂, CON(C₁-C₆ alkyl)₂, and NH(CO)—C₁-C₆ alkyl;
R′ is selected from the group comprising hydrogen, C₁-C₆ straight chain and branched alkyl, and C₃-C₆ cycloalkyl, and
R″ is selected from the group comprising hydrogen, benzyl, and acetyl, or a composition comprising said compound of formula I.

In a further aspect, the invention provides a method for the treatment of leaky gut, a method for the treatment of leaky gut syndrome, or a method for the improvement of at least one symptom associated with leaky gut syndrome, the method comprising the step of administering to the subject an effective amount of phenylcapsaicin (V), or any pharmaceutically acceptable salt or solvate thereof, to a subject.

In some embodiments, the invention provides a method for the treatment of leaky gut, a method for the treatment of leaky gut syndrome, or a method for the improvement of at least one symptom associated with leaky gut syndrome, the method comprising the step of administering to the subject a composition comprising an effective amount of phenylcapsaicin (V), or any pharmaceutically acceptable salt or solvate thereof to a subject.

In a further aspect, the invention provides the use of a compound of formula I, or any pharmaceutically acceptable salt or solvate thereof, to inhibit quorum sensing, to inhibit quorum sensing in the gut, to treat leaky gut, to treat leaky gut syndrome, or to improve at least one symptom associated with leaky gut syndrome.

In a further aspect, the invention provides the use of a composition comprising an effective amount of a compound of formula I, to inhibit quorum sensing, to inhibit quorum sensing in the gut, to treat leaky gut, to treat leaky gut syndrome, or to improve at least one symptom associated with leaky gut syndrome.

In a further aspect, the invention provides the use of phenylcapsaicin (V), or any pharmaceutically acceptable salt or solvate thereof, to inhibit quorum sensing, to inhibit quorum sensing in the gut, to treat leaky gut, to treat leaky gut syndrome, or to improve at least one symptom associated with leaky gut syndrome.

In a further aspect, the invention provides the use of a composition comprising phenylcapsaicin (V), to inhibit quorum sensing, to inhibit quorum sensing in the gut, to treat leaky gut, to treat leaky gut syndrome, or to improve at least one symptom associated with leaky gut syndrome.

In a further aspect, the invention provides the use of a compound of formula I, or a pharmaceutically acceptable salt or solvate thereof, or a composition comprising said compound of formula I, for inhibition of quorum sensing. Said quorum sensing may be undesired quorum sensing. Said quorum sensing may be in the gut. The use may be therapeutic. The use may be non-therapeutic, such as non-therapeutic prevention of biofilm.

The invention shall not be limited to the shown embodiments and examples. While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is to be understood that various alternatives to the embodiments described herein can be employed in practicing the disclosure.

It is to be understood that every embodiment of the disclosure can optionally be combined with any one or more of the other embodiments described herein.

It is to be understood that each component, compound, or parameter disclosed herein is to be interpreted as being disclosed for use alone or in combination with one or more of each and every other component, compound, or parameter disclosed herein. It is further to be understood that each amount/value or range of amounts/values for each component, compound, or parameter disclosed herein is to be interpreted as also being disclosed in combination with each amount/value or range of amounts/values disclosed for any other component(s), compound(s), or parameter(s) disclosed herein, and that any combination of amounts/values or ranges of amounts/values for two or more component(s), compound(s), or parameter(s) disclosed herein are thus also disclosed in combination with each other for the purposes of this description. Any and all features described herein, and combinations of such features, are included within the scope of the present invention provided that the features are not mutually inconsistent.

It is to be understood that each lower limit of each range disclosed herein is to be interpreted as disclosed in combination with each upper limit of each range disclosed herein for the same component, compound, or parameter. Thus, a disclosure of two ranges is to be interpreted as a disclosure of four ranges derived by combining each lower limit of each range with each upper limit of each range. A disclosure of three ranges is to be interpreted as a disclosure of nine ranges derived by combining each lower limit of each range with each upper limit of each range, etc. Furthermore, specific amounts/values of a component, compound, or parameter disclosed in the description or an example is to be interpreted as a disclosure of either a lower or an upper limit of a range and thus can be combined with any other lower or upper limit or a range or specific amount/value for the same component, compound, or parameter disclosed elsewhere in the application to form a range for that component, compound, or parameter.

### EXAMPLES

### Example 1—Inhibition of Quorum Sensing Activity by Phenycapsaicin (V) Using Chromobacterium Violaceum CV026

Phenylcapsaicin (V) and capsaicin were assayed for their quorum sensing-inhibitory activity using chromobacterium violaceum CV026.

Chromobacterium violaceum CV026 is a violacein negative, double mini-Tn5 mutant from C. violaceum (ATCC 31532) that was used to identify quorum sensing inhibition. Chromobacterium violaceum CV026 is deficient in the autoinducer acyl homoserine lactone synthetase and therefore requires exogenous addition of N-hexanoyl homoserine lactone (C6-HSL) to undergo quorum sensing and produce a natural antibiotic called violacein, which is a water-insoluble purple pigment with antibacterial activity.

Method:

A stock solution of 1000 ug of compound V and capsaicin was prepared by dissolving 1000 μg in 100 μl of ethanol and 900 μl of sterile water. The stock solutions were sequentially diluted with double volumes of solvent (10% aqueous ethanol solution) to yield 500/250/125/62.5/31.25/15.625/7.81 μg/ml test solutions.

Chromobacterium violaceum CV026 was used to determine the anti-quorum sensing activity. Inducer N-hexanoyl homoserine lactone (C6-HSL) was purchased from Sigma-Aldrich. CV026 was cultivated in Luria-Bertani (LB) medium (LB, 5 g yeast extract, 10 g tryptone, 5 g NaCl, 1 L water) at 30° C. overnight under aerobic conditions. A standard disc-diffusion assay was used to detect anti-quorum sensing activity of V and capsaicin by making use of double layer culture plates. 15 ml LB medium (2.5% agar) was overlaid with 15 ml LB (1.0% agar) containing 50 μl C. violaceum CV026 with 20 μg/ml-1 kanamycin and 50 ng/ml C6-AHL. Test concentrations of V and capsaicin were added onto sterile disks (8 mm diameter) and placed on the agar. The plates were then incubated overnight at 30° C. and quorum sensing inhibition was detected as a less colored/colourless zone around the disk, where viable cells, indicative of growth but quorum sensing inhibition, were present. Measurements were made from the outer edge of the discs to the edge of the zones of anti-quorum sensing inhibition. The formulation solvent (10% aqueous ethanol) was used as the negative control.

Results:

The biosensor strain, C. violaceum CV026, is a mutant of the wild type strain and is unable to produce its own AHL signal. It only responds to exogenous active signal molecules and produces a purple color, violacein. Loss of this purple color around the disk when incubated with exogenous AHL is thus indicative of QS inhibition by the test compounds. More specifically, the zone (mm past the disk) of QS inhibition results in an opaque disc with no purple color but bacterial growth. Inhibition of bacterial growth (antibacterial activity) is indicated by a transparent zone of inhibition containing no bacteria. The results are shown below in Table 1, wherein QS denotes quorum sensing.

TABLE 1
QS inhibition vs antibacterial control
for phenylcapsaicin (V) and capsaicin

| | |
|---|---|
| Phenylcapsaicin (V) | Capsaicin |

| Conc (μg/mL) | QS | Antibacterial | QS | Antibacterial |
|---|---|---|---|---|
| 500 | NO | 10 | NO | 11 |
| 250 | NO | 9 | NO | 14 |
| 125 | NO | 5 | NO | 9 |
| 62.5 | 7 | 3 | NO | 8 |
| 31.13 | 11 | 0 | 0 | 0 |
| 15.63 | 9 | 0 | 0 | 0 |
| 7.81 | 3 | 0 | 0 | 0 |

Zones: >10 mm strong; 5 mm-10 mm moderate; 1 mm-5 mm weak; NO=not observable

Discussion/Conclusion:

In this CV026 bacteria quorum sensing inhibition assay, V was shown to be a quorum sensing inhibitor in a dose dependent manner at doses lower than the sub-lethal antibacterial doses observed. Specifically, compound V at 61.5/31.25/15.625/7.81 μg/ml doses showed strong to weak quorum sensing inhibiting activity in a dose responsive manner, while showing antibacterial activity at higher doses. (500/250/125 μg/ml).

The natural compound capsaicin did not exhibit quorum sensing inhibiting activity at non antibacterial doses (31.25/15.625/7.81 μg/ml), lending further credibility to the unanticipated nature of the quorum sensing inhibiting activity for the compounds for use in a method for the treatment of leaky gut, a method for the treatment of leaky gut syndrome, or a method for the improvement of at least one symptom associated with leaky gut syndrome according to the invention.

This result indicates that compound V, and by extension the other compounds for use in a method for the treatment of leaky gut, a method for the treatment of leaky gut syndrome, or a method for the improvement of at least one symptom associated with leaky gut syndrome according to the invention, have a novel, unanticipated mechanism of action at sub-lethal antibacterial doses that reduces the pathogenicity of bacteria and hence shows an improvement in GI inflammatory conditions, such as leaky gut syndrome.

### Example 2—Investigation of Antibacterial Effects of Phenylcapsaicin (V)

The antibacterial effects of phenylcapsaicin in vitro were investigated to determine any bactericidal activity of phenylcapsaicin (V) against three bacterial pathogens.

The study included salmonella, campylobacter and listeria. Four different concentrations of phenylcapsaicin were tested: 19 ppm, 56 ppm, 167 ppm, and 500 ppm.

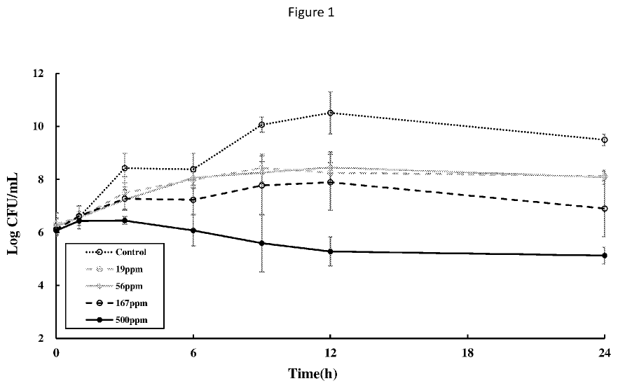

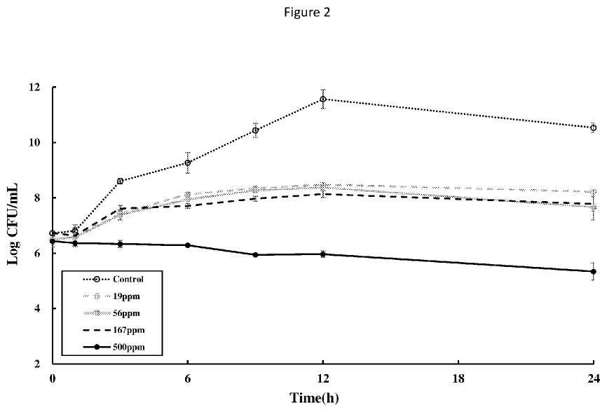

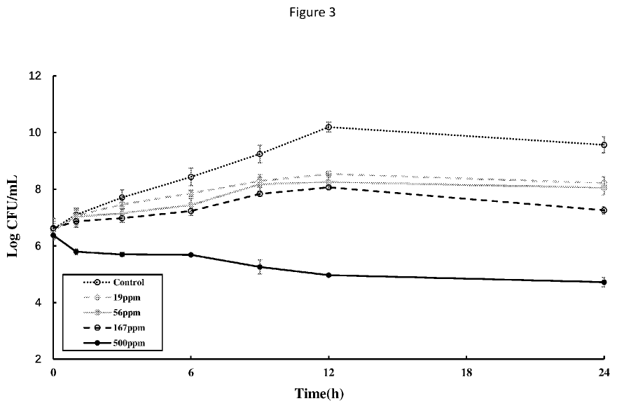

The results of the study are shown in FIGS. 1, 2, and 3, and clearly show that non and sublethal doses for phenylcapsaicin and its derivatives can be experimentally determined and used to define and deliver an effective dose of compounds of the said invention for the treatment of leaky gut and leaky gut syndrome by inhibiting quorum sensing in gut bacteria.

### Example 3—Inhibition of Biofilm Formation Using Intestinal Pathogenic P. Aeruginosa (ATCC® 15442)

Background:

Quorum sensing is a regulatory mechanism that many bacterial species use to control expression of gene circuits in a population-dependent manner. Quorum sensing uses small diffusible molecules called autoinducers to monitor and mediate the size of a bacterial population. Autoinducers produced by bacteria diffuse out and accumulate in the surrounding environment, and once a threshold concentration has been reached, they diffuse back into the bacteria and regulate the transcription of specific genes, many of which can turn an otherwise benign bacteria into a pathogenic one. Increasing evidence indicates that bacterial quorum sensing is involved in the regulation of diverse biological processes such as virulence factor gene expression, sporulation, biofilm formation and motility which often result in GI distress symptoms commonly associated with leaky gut syndrome.

In commercially important gram-negative bacteria, exemplified in this assay by P. aeruginosa which is a common encapsulated, Gram-negative, rod-shaped bacterium, N-acyl homoserine lactones (AHL) are most commonly used as the auto-inducers. The quorum sensing system can be interfered with in a number of ways. Inactivating the quorum sensing system of a bacterial pathogen can result in a significant decrease in virulence factor production. One measure of quorum sensing inhibition is the inhibition of biofilm formation by pathogenic bacteria which is the focus of this assay.

Aim:

The aim of the study was to assay phenylcapsaicin (V) and capsaicin for their biofilm formation inhibition using Pseudomonas aeruginosa (ATCC® 15442) bacteria.

Method:

A stock solution of 1000 ug of V and capsaicin was prepared by dissolving 1000 μg in 100 μl of ethanol and 900 μl of sterile water. The stock solutions were sequentially diluted with double volumes of solvent (10% aqueous ethanol solution) to yield 500/125//31.25/7.81 μg/ml test solutions.

P. aeruginosa (ATCC® 15442) was used to determine the biofilm inhibition activity. Inducer N-hexanoyl homoserine lactone (C6-HSL) was purchased from Sigma-Aldrich. ATCC® 15442 was cultivated in Luria-Bertani (LB) medium (LB, 5 g yeast extract, 10 g tryptone, 5 g NaCl, 1 L water) at 30° C. overnight under aerobic conditions.

Crystal Violet Staining Assay

Biofilm formation was quantified with modifications using the method described by Stepanovic et al. (Journal of Microbiological Methods, 2000, 40(2), 175-19) with modifications. Using a 96-well microplate duplicate the test solutions were filled at the above selected doses. Cultures were grown for 18 hr in LB at 30° C. and adjusted to OD 0.1 at 600 nm. 20 μL of the culture were added into each well and the remainder of the volume to 200 μL per well was filled with LB. Unused external wells were filled with 200 μL of sterile water to prevent evaporation.

After 24 hr of incubation, supernatants were discarded, and the wells were washed three times with 200 μL of sterile phosphate buffered saline (PBS). Bacteria adhered to the wells were fixed with 200 μL of methanol for 15 min, the methanol was discarded, and the plates dried at 25° C.

Each well was stained with 200 μL of 0.3% (w/v) crystal violet solution for 5 min at 25° C. Crystal violet excess was pipetted out, and the wells were rinsed five times with sterile PBS. 200 μL of 33% (v/v) glacial acetic acid were added to the wells for 30 min to solubilise the dye adhered to the cell walls. Optical density (OD) was measured at 595 nm using a microplate spectrophotometer as a direct measure of biofilm formation. P. aeruginosa in 3% DMSO will be used as the negative control.

Results:

Table 2 shows the effect of phenylcapsaicin and capsaicin on biofilm formation by P. aeruginosa (ATCC® 15442), a bacterium with a well-characterised quorum sensing system.

TABLE 2
effect of phenylcapsaicin and capsaicin
on biofilm formation by P. aeruginosa

| P. aeruginosa | Phenylcapsaicin | Capsaicin |
|---|---|---|
| Conc (μg/mL) | Biofilm (OD 595 nm) | Biofilm (OD 595 nm) |
| Control | 6.7^{a} | 6.8^{a} |
| 500 | — | — |
| 125 | 1.9^{b} | 7.2^{a} |
| 31.125 | 1.3^{b} | 7.0^{a} |
| 7.81 | 1.7^{b} | 7.1^{a} |

— = growth interference

Based on the OD intensity obtained in the crystal violet staining assay (CVSA), the biofilm formed by P. aeruginosa (ATCC® 15442) at 30° C. was significantly reduced by phenylcapsaicin at concentrations less than 125 ug/ml, although no dose-dependency was observed. Capsaicin, at the same concentrations, did not show any reduction of biofilm formation.

Both compounds showed growth interference at the 500 ug/ml concentration.

The biomass formed by the control at 30° C. was adequate for biofilm, studies but the biofilm at 37° C. was ˜OD 2.9 and judged inadequate for biofilm reduction quantification.

Control represents bacterial growth in LB with 3% aq DMSO carrier blank.

The values shown are the mean of triplicate assays at OD 595 nm.

Values followed by different letters differ at 5% probability (P<0.05) by Tukey's test.

Discussion/Conclusion:

In this biofilm inhibition assay, phenylcapsaicin was shown to be a potent inhibitor of biofilm formation in P. aeruginosa (ATCC® 15442) bacteria. Capsaicin did not exhibit any biofilm formation inhibition at any dose tested. This result shows that phenylcapsaicin may have a novel, unanticipated mechanism of action at sub-lethal antibacterial doses that reduces the pathogenicity of bacteria and hence shows an improvement in GI inflammatory conditions, such as leaky gut syndrome.

### Example 4—Inhibition of Biofilm Formation Using Pathogenic Enterohemorrhagic Bacteria Escherichia coli O157:H7

Based on the same background as Example 3, the aim of this study was to assay phenylcapsaicin (V) and capsaicin for their biofilm formation inhibition using pathogenic enterohemorrhagic bacteria Escherichia coli O157:H7.

Method:

A stock solution of 1000 μg of phenylcapsaicin and capsaicin was prepared by dissolving 1000 μg in 100 μL of ethanol and 900 μL of sterile water. The stock solutions were sequentially diluted with double and then quadruple volumes of solvent (10% aqueous ethanol solution) to yield 500/125/31.25 (32)/7.81 (8) μg/ml test solutions.

E. coli O157:H7 was used to determine the biofilm inhibition activity. Inducer N-hexanoyl homoserine lactone (C6-HSL) was purchased from Sigma-Aldrich. E. coli O157:H7 was cultivated in Luria-Bertani (LB) medium (LB, 5 g yeast extract, 10 g tryptone, 5 g NaCl, 1 L water) at 37° C. overnight under aerobic conditions.

Crystal violet staining assay was performed as in Experiment 3, using E. coli O157:H7 in 3% DMSO as the control.

Results:

Table 3 shows the effect of phenylcapsaicin and capsaicin on biofilm formation by E. coli O157:H7.

TABLE 3
effect of phenylcapsaicin and capsaicin on
biofilm formation by E. coli O157:H7

| E. coli O157:H7 | Phenylcapsaicin | Capsaicin |
|---|---|---|
| Conc (μg/mL) | Biofilm (OD 595 nm) | Biofilm (OD 595 nm) |
| Control | 5.5^{a} | 5.5^{a} |
| 500 | — | — |
| 125 | 1.9^{b} | 5.1^{a} |
| 32 | 2.4^{b} | 5.8^{a} |
| 8 | 4.9^{b} | 5.9^{a} |

— = growth interference

Based on the OD intensity obtained in the crystal violet staining assay (CVSA), the biofilm formed by E. coli O157:H7 at 30° C. was significantly reduced by phenylcapsaicin at concentrations of 125 and 32 μg/ml, but with no effect at the 8 μg/ml lowest dose tested.

Capsaicin, at the same concentrations, did not show any reduction of biofilm formation.

Both compounds showed growth interference at the 500 μg/ml concentration.

As with P. aeruginosa bacteria (Example 3), biomass formed by the control at 30° C. was adequate for biofilm studies but the biofilm at 37° C. was ˜OD 2.9 and judged inadequate for biofilm reduction quantification.

The Control represents bacterial growth in LB with 3% aq. DMSO carrier blank.

The values shown are the mean of triplicate assays at OD 595 nm.

Values followed by different letters differ at 5% probability (P<0.05) by Tukey's test.

Discussion/Conclusion:

In this biofilm inhibition assay, phenylcapsaicin was shown to be a good inhibitor of biofilm formation in E. coli O157:H7 bacteria at moderate concentrations of 125 and 32 μg/ml but not at the lowest rate tested 8 μg/ml. Capsaicin did not exhibit any biofilm formation inhibition at any dose tested. This result shows that phenylcapsaicin continues to have a novel, unanticipated mechanism of action at sub-lethal antibacterial doses that reduces the pathogenicity of various bacteria and likely to show an improvement in GI inflammatory conditions, such as leaky gut syndrome.

### Example 5—Inhibition of Biofilm Formation Using Salmonella enterica Subsp. Enterica Serovar Paratyphi C (ATCC® BAA 1714™)

Based on the same background as Examples 3 and 4, the aim of this study was to assay phenylcapsaicin (V) and capsaicin for their biofilm formation inhibition using Salmonella enterica subsp. enterica serovar Paratyphi C (ATCC® BAA 1714™) bacteria strain.

Method:

A stock solution of 1000 μg of phenylcapsaicin and capsaicin was prepared by dissolving 1000 μg in 100 μl of ethanol and 900 μl of sterile water. The stock solutions were sequentially diluted with double and then quadruple volumes of solvent (10% aqueous ethanol solution) to yield 500/125/31.25 (32)/7.81 (8) μg/ml test solutions.

ATCC® BAA 1714™ was used to determine the biofilm inhibition activity. Inducer N-hexanoyl homoserine lactone (C6-HSL) was purchased from Sigma-Aldrich. ATCC® BAA 1714™ was cultivated in Luria-Bertani (LB) medium (LB, 5 g yeast extract, 10 g tryptone, 5 g NaCl, 1 L water) at 37° C. overnight under aerobic conditions.

Crystal violet staining assay was performed as in Experiment 3, using ATCC® BAA 1714™ in 3% DMSO as the control.

Results:

Table 4 shows the effect of phenylcapsaicin and capsaicin on biofilm formation by ATCC® BAA 1714™.

TABLE 4
effect of phenylcapsaicin and capsaicin on biofilm formation
by ATCC ® BAA 1714 ™

| Salmonella enterica | | |
|---|---|---|
| subsp. | | |
| enterica serovar | | |
| Paratyphi C (ATCC ® | | |
| BAA 1714 ™) | Phenylcapsaicin | Capsaicin |
| Conc (μg/mL) | Biofilm (OD 595 nm) | Biofilm (OD 595 nm) |
| Control | 8.5^{a} | 9.0^{a} |
| 500 | — | — |
| 125 | — | — |
| 31.125 | 3.2^{b} | 8.1^{a} |
| 7.81 | 2.9^{b} | 8.5^{a} |

— = growth interference

Based on the OD intensity obtained in the crystal violet staining assay (CVSA), the biofilm formed by ATCC® BAA 1714™ at 30° C. was significantly reduced by phenylcapsaicin at concentrations of 32 and 8 μg/ml which was the lowest dose tested. Capsaicin, at the same concentrations, did not show any reduction of biofilm formation.

Both compounds showed growth interference at the 500 and 125 μg/ml concentration.

As with previous assays the biomass formed by the control at 30° C. was adequate for biofilm studies but the biofilm at 37° C. was ˜OD 2.6 and judged inadequate for biofilm reduction quantification.

The Control represents bacterial growth in LB with 3% aq DMSO carrier blank. The values shown are the mean of triplicate assays at OD 595 nm. (SD=/−0.7) Values followed by different letters differ at 5% probability (P<0.05) by Tukey's test.

Discussion/Conclusion:

In this biofilm inhibition assay, phenylcapsaicin was shown to be a good inhibitor of biofilm formation in ATCC® BAA 1714™ bacteria at low concentrations of 32 and 8 μg/ml which was the lowest rate tested. Capsaicin did not exhibit any biofilm formation inhibition at any dose tested. This result shows that phenylcapsaicin continues to have a novel, unanticipated mechanism of action at sub-lethal antibacterial doses that reduces the pathogenicity of various bacteria and likely to show an improvement in GI inflammatory conditions, such as leaky gut syndrome.

### Example 6—Inhibition of Biofilm Formation Using Salmonella enterica Subsp. Enterica Serovar Paratyphi C (ATCC® BAA1714™) with Different Capsaicyn Compounds

Based on the same background as Example 3, the aim of this study was to assay capsaicin, phenylcapsaicin (V) and three other capsaicyns, shown below and denoted a-c, for their biofilm formation inhibition using Salmonella enterica subsp. enterica serovar Paratyphi C (ATCC® BAA1714™ bacteria strain.

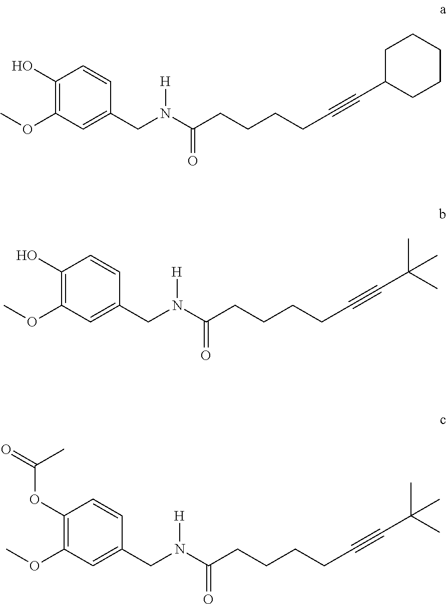 [SMILES: COc1cc(CNC(=O)CCCCC#CC2CCCCC2)ccc1O ; COc1cc(CNC(=O)CCCCC#CC(C)(C)C)ccc1O ; COc1cc(CNC(=O)CCCCC#CC(C)(C)C)ccc1OC(C)=O]

Method:

Since phenylcapsaicin and capsaicin both showed growth interference (antibacterial activity) at the 500 μg/ml concentration in Example 5, this high rate was eliminated from this assay.

A stock solution of 500 μg of phenylcapsaicin, capsaicin, compound a, compound b, compound c, was prepared by dissolving 500 μg in 100 μl of ethanol and 900 μl of sterile water. The stock solutions were sequentially diluted with quadruple volumes of solvent (10% aqueous ethanol solution) to yield 125/31.25 (32)/7.81 (8)/1.95 (2) μg/ml test solutions.

ATCC® BAA1714™ was used to determine the biofilm inhibition activity. Inducer N-hexanoyl homoserine lactone (C6-HSL) was purchased from Sigma-Aldrich. ATCC® BAA1714™ was cultivated in Luria-Bertani (LB) medium (LB, 5 g yeast extract, 10 g tryptone, 5 g NaCl, 1 L water) at 37° C. overnight under aerobic conditions.

Crystal violet staining assay was performed as in Experiment 3, using ATCC® BAA1714™ in 3% DMSO as the control.

Results:

Table 5 shows the effect of phenylcapsaicin, capsaicin, compound a, compound b, and compound c, on biofilm formation by ATCC® BAA 1714™.

TABLE 5
effect of phenylcapsaicin, capsaicin, and each of compounds a-c on
biofilm formation by ATCC ® BAA 1714 ™

| Salmonella enterica | | | | | |
|---|---|---|---|---|---|
| subsp. | | | | | |
| enterica serovar | Phenyl- | | | | |
| Paratyphi C | capsaicin | Capsaicin | a | b | c |
| (ATCC ® BAA | Biofilm | Biofilm | Biofilm | Biofilm | Biofilm |
| 1714 ™) | (OD | (OD | (OD | (OD | (OD |
| Conc (μg/mL) | 595 nm) | 595 nm) | 595 nm) | 595 nm) | 595 nm) |
| Control | 8.8 | 9.1 | 8.8 | 8.8 | 8.9 |
| 125 | — | — | 8.2 | 8.8 | 8.7 |
| 32 | 3.4 | 8.2 | 7.1 | 8.2 | 8.5 |
| 8 | 2.8 | 8.6 | 6.8 | 7.9 | 8.6 |
| 2 | 2.5 | 8.2 | 6.5 | 8.1 | 8.0 |

— = growth interference

Based on the OD intensity obtained in the crystal violet staining assay (CVSA), the biofilm formed by ATCC® BAA1714™ at 30° C. was once again significantly reduced by phenylcapsaicin at concentrations of 32, 8 and at the new low dose of 2 μg/ml which was the lowest dose tested. The 125 μg/ml dose still showed antibacterial interference with the biofilm readings.

Capsaicin did not show reduction biofilm formation at any dose tested. The 125 μg/ml dose still showed antibacterial interference with the biofilm readings.

Phenylcapsacin analogue a showed modest biofilm formation inhibition activity, at 2 μg/ml dose. Compounds b and c did not show biofilm formation inhibition activity.

As with previous assays the biomass formed by the control at 30° C. was adequate for biofilm studies but the biofilm at 37° C. was ˜OD 2.6 and judged inadequate for biofilm reduction quantification.

The Control represents bacterial growth in LB with 3% aq DMSO carrier blank. The values shown are the mean of triplicate assays at OD 595 nm (SD+/−0.66). Values followed by different letters differ at 5% probability (P<0.05) by Tukey's test.

Discussion/Conclusion:

In this biofilm formation inhibition assay, phenylcapsaicin is again shown to be a good inhibitor of biofilm formation in ATCC® BAA1714™ bacteria at concentrations of 32, 8 and the new lower 2 μg/ml.

Of the three phenylcapsaicin analogues tested, only a showed some modest biofilm formation inhibition activity, approximating 33% of the activity of phenylcapsaicin at 2 μg/ml dose. This result can be used to develop a QSAR model for biofilm inhibition with the phenylcapsaicin structure.

The results indicate that different capsaicyns have a novel, unanticipated mechanism of action at sub-lethal antibacterial doses that reduces the pathogenicity of various bacteria and likely to show an improvement in GI inflammatory conditions, such as leaky gut syndrome.

1. A method for the treatment of leaky gut, leaky gut syndrome, or increased intestinal permeability, the method comprising the step of administering to a subject an effective amount of a compound of formula I, or a pharmaceutically acceptable salt or solvate thereof, 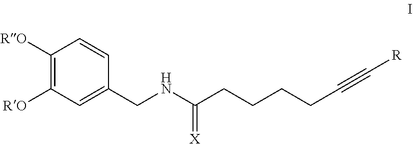
wherein
X is selected from oxygen and sulphur,
R is selected from the group comprising cyclohexyl, phenyl and substituted phenyl;
wherein said substituted phenyl is substituted in any one or more positions with 1-5 identical or different substituents selected from the group comprising fluoro; chloro; bromo; iodo, cyano, nitro, trifluoromethyl, C₁-C₆ straight chain and branched alkoxy, C₁-C₆ sulfoxy, —S—C₁-C₆ alkyl, C₁-C₆ straight chain and branched alkyl, alkenyl, and alkynyl C₂-C₆ straight chain and branched alkenyl, C₂-C₆ straight chain and branched alkynyl, C₁-C₆ fluoroalkyl, chloroalkyl, bromoalkyl, and iodoalkyl, COO—C₁-C₆ alkyl, O(CO)—C₁-C₆ alkyl, NH—C₁-C₆ alkyl, N(C₁-C₆ alkyl)₂, CON(C₁-C₆ alkyl)₂, and NH(CO)—C₁-C₆ alkyl:
R′ is selected from the group comprising hydrogen, C₁-C₆ straight chain and branched alkyl, and C₃-C₆ cycloalkyl, and
R″ is selected from the group comprising hydrogen, benzyl, and acetyl, or a composition comprising said compound of formula I.

2. The method according to claim 1, wherein R is selected from the group comprising phenyl and substituted phenyl.

3. The method according to claim 1 for the treatment of a subclinical increase in gut permeability in a subject who experiences one or more symptoms associated with leaky gut syndrome and who displays a significant deviation in the level of at least one biomarker associated with leaky gut, leaky gut syndrome and/or intestinal hyperpermeability compared to a normal level of the at least one biomarker.

4. The method according to claim 1, wherein X is oxygen.

5. The method according to claim 1, wherein X is sulphur.

6. The method according to claim 1, wherein R′ is methyl.

7. The method according to claim 1, wherein R″ is hydrogen.

8. The method according to claim 1, wherein the compound is phenylcapsaicin of structure V.

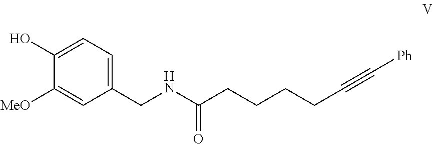 [SMILES: COc1cc(CNC(=O)CCCCC#Cc2ccccc2)ccc1O]

9. The method according to claim 1, administering a composition comprising the compound or pharmaceutically acceptable salt or solvate thereof of formula I, to the subject.

10. The method according to claim 3, wherein the at least one biomarker is zonulin.

11. The method according to claim 1, wherein the subject has been diagnosed with leaky gut.

12. The method according to claim 1, wherein the subject has not been diagnosed with celiac disease, IBD, ulcerative colitis, or Crohn's disease.

13. (canceled)

14. The method according to claim 1, wherein the method comprises the step of administering the compound to a subject by oral and/or intraperitoneal administration.

15. The method according to claim 1, wherein the method comprises the step of administering the compound to a subject by oral administration.

16. The method according to claim 15, wherein the compound is formulated for oral administration.

17. The method according to claim 10, wherein the subject has been diagnosed with leaky gut.

18. The method according to claim 3, wherein the subject has not been diagnosed with celiac disease, IBD, ulcerative colitis, or Crohn's disease.
